(19) United States
(12) Patent Application Publication
(10) Pub. No.: US 2013/0110150 A1
(43) Pub. Date: May 2, 2013

(54) ELECTROACUPUNCTURE NEEDLE AND ELECTROACUPUNCTURE PLATFORM

(71) Applicant: Korea Advanced Institute of Science & Technology, Daejeon (KR)

(72) Inventors: Hoi-Jun YOO, Daejeon (KR); Hyungwoo Lee, Daejeon (KR)

(73) Assignee: Korea Advanced Institute of Science & Technology, Daejeon (KR)

(21) Appl. No.: 13/659,449

(22) Filed: Oct. 24, 2012

(30) Foreign Application Priority Data
Oct. 26, 2011 (KR) 10-2011-0109872

(51) Int. Cl. A61N 1/05

(52) U.S. Cl. 606/189

(57) ABSTRACT

An electroacupuncture needle may be provided that includes: a body which includes at least one boundary line having discontinuous electrical conductivity; and an end which is formed to have a pointed shape allowing the electroacupuncture needle to be easily inserted into the skin of a subject.

The present application claims priority under 35 U.S.C. §119 to Korean Patent Application No. 10-2011-0109872, filed on Oct. 26, 2011, the entirety of which is hereby incorporated by reference.

### FIELD

The present invention relates to an electroacupuncture needle and an electroacupuncture platform including the same.

### DESCRIPTION OF RELATED ART

Human body, a part of nature, is affected by electricity and bioelectric current is generated and circulates in a whole body. Bioelectricity is a kind of electrical phenomena occurring in our body. It was known that electricity with a voltage of 5 to 6 V flows in a body at birth and electricity with a voltage less than 2.5 V flows when biologically aged. With regard to this, abnormality of the bioelectric current in our body causes diseases. Medical treatment devices are now being variously employed in an acupuncture therapy of oriental medicine and new clinical treatments of a modern science.

Electroacupuncture has been developed as a treatment using the bioelectric current. In 1816, a French, Berlioz has announced a method for curing a neuralgia through an acupuncture therapy using flowing current. In 1825, a French doctor, Sarlandier has treated gout and a nervous system by applying the electroacupuncture. In 1921, an English, E. A. Golden has established a base of a modern electroacupuncture therapy. Subsequently, researches have been being actively devoted to the electroacupuncture in China, Japan, Germany, etc.

Recently, as much attention has been worldly paid to an alternative medicine, an electroacupuncture treatment device is now widely being used in non-medical field like fatness, hair-removal and the like, particularly, a beauty field as well as a medical field. With regard to this, for more effective treatment, it is necessary to reduce electrical resistance and to provide electrical stimulation capable of controlling an electric current path between the electroacupuncture needles.

### SUMMARY

One embodiment is an electroacupuncture needle. The electroacupuncture needle includes: a body which includes at least one boundary line having discontinuous electrical conductivity; and an end which is formed to have a pointed shape allowing the electroacupuncture needle to be easily inserted into the skin of a subject.

Another embodiment is an electroacupuncture needle. The electroacupuncture needle includes: a body which has unevenness formed on the surface thereof; and an end which is formed to have a pointed shape allowing the electroacupuncture needle to be easily inserted into the skin of a subject.

Further another embodiment is an electroacupuncture platform. The electroacupuncture platform includes: a current supply supplying current for electrical stimulation; and a plurality of electroacupuncture needles electrically connected to one end of the current supply. At least one of the plurality of the electroacupuncture needles is according to the embodiment of the present invention.

### BRIEF DESCRIPTION OF THE DRAWINGS

FIG. 1 shows that an electroacupuncture needle according to an embodiment of the present invention is inserted into the skin of an examinee;

FIG. 2 shows that the electroacupuncture needle is implemented by using an insulation material in accordance with the embodiment of the present invention;

FIG. 3 shows that the electroacupuncture needle is implemented by plating in accordance with the embodiment of the present invention;

FIG. 4 shows that the electroacupuncture needle is implemented by changing a constituent metallic material in accordance with the embodiment of the present invention;

FIG. 5 shows that the electroacupuncture needle having a textured surface is implemented in accordance with the embodiment of the present invention;

FIG. 6 shows that the electroacupuncture needle is implemented by combining the methods of FIGS. 2 to 5 in accordance with the embodiment of the present invention;

FIG. 7 shows a current path induction effect of an electroacupuncture platform using two electroacupuncture needles according to the embodiment of the present invention;

FIG. 8 shows a current path induction effect of an electroacupuncture platform using four electroacupuncture needles according to the embodiment of the present invention;

FIG. 9 shows a current path induction effect of an electroacupuncture platform including two general electroacupuncture needles and two electroacupuncture needles according to the embodiment of the present invention;

FIG. 10A shows how to apply mutually different electrical stimulation patterns to a plurality of electrical conductive surfaces of one electroacupuncture needle in accordance with the embodiment of the present invention; and

FIG. 10B shows how at least one electrical conductive surface of one electroacupuncture needle is used as a ground plane in accordance with the embodiment of the present invention.

### DETAILED DESCRIPTION

Hereafter, an exemplary embodiment of the present invention will be described in detail with reference to the accompanying drawings. The shapes and sizes and the like of components of the drawings are exaggerated for clarity of the description. It is noted that the same reference numerals are used to denote the same elements throughout the drawings. In the following description of the present invention, the detailed description of known functions and configurations incorporated herein is omitted when it may make the subject matter of the present invention unclear.

The present invention is based on a principle in which electric charges are concentrated either on a cusp projected from a surface or on a boundary in which electrical conductivity is discontinuously changed. Therefore, a body of the electroacupuncture needle according to the embodiment of the present invention may include a boundary line having discontinuous electrical conductivity. Otherwise, the surface of the body may be textured. Here, the unevenness may function as a cusp on which electric charges are concentrated on the surface of the electroacupuncture needle.

FIG. 1 shows that an electroacupuncture needle 100 according to an embodiment of the present invention is inserted into the skin of a subject. Here, it is shown that the electroacupuncture needle 100 includes at least one body part and an end part. The body includes a boundary line in which electrical conductivity is changed. The end part is formed to have a pointed shape allowing the electroacupuncture needle to be easily inserted into the skin of a subject. Additionally, the electroacupuncture needle 100 may include a boundary line having discontinuous electrical conductivity and/or a surface unevenness.

Here, the subject is an object in which electrical stimulation can be performed by the electroacupuncture needle. While a human is mentioned as an object in which the electrical stimulation is performed in the present specification, the object is not limited to a human. It should be noted that any object allowing the electrical stimulation to be performed by means of electricity can be employed. For example, the subject may include an animal or plant species as well as a human.

The electroacupuncture needle 100 is inserted into the skin of the subject and functions as a stimulation electrode which introduces an electrical stimulation pulse into a human body. As shown in FIG. 1, the electroacupuncture needle 100 may have a shape like a needle having conductivity which can be used in a general electroacupuncture treatment.

Here, the end of the electroacupuncture corresponds to a pointed portion which is the most deeply positioned in the skin of the subject in FIG. 1. The boundary line of the body, which has discontinuous electrical conductivity, may be, as shown in FIG. 1, positioned at a portion contacting with the inside of the skin of the subject. In other words, the boundary line may be formed between a material denoted by a reference numeral 110 and a material denoted by a reference numeral 120 in FIG. 1. That is, the boundary line having discontinuous electrical conductivity may be formed between the first material 110 and the second material 120, both of which have mutually different electrical conductivities. When the surface of the electroacupuncture needle 100 is textured, the textured surface may be also positioned at a portion contacting with the inside of the skin of the subject.

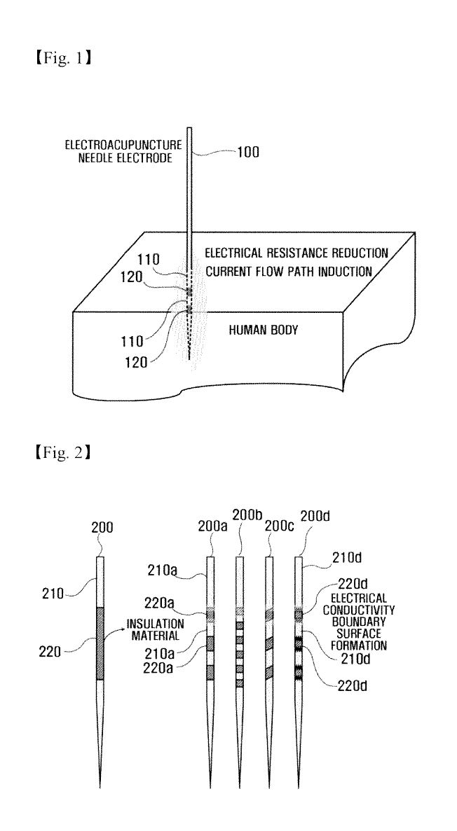

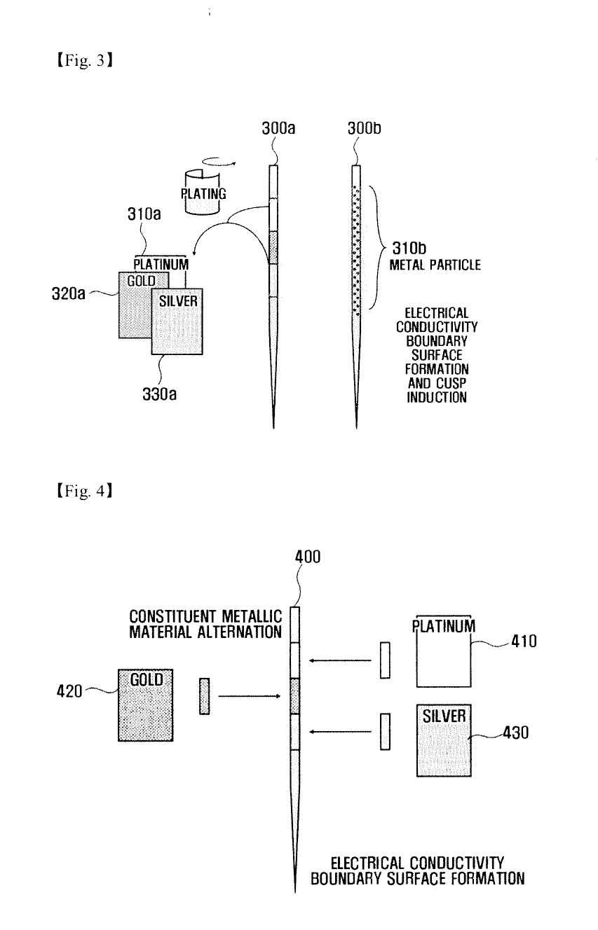

In the electroacupuncture needle 100, the boundary line having discontinuous electrical conductivity may be implemented in various methods. Some of the methods will be described with reference to FIGS. 2 to 4.

FIG. 2 shows that electroacupuncture needle 200, 200a, 200b, 200c and 200d is implemented by using an insulation material 220 in accordance with the embodiment of the present invention. In FIG. 2, the electroacupuncture needle 200, 200a, 200b, 200c and 200d may be formed with a material 210 having first electrical conductivity. Some portions of the body of the electroacupuncture needle 200, 200a, 200b, 200c and 200d may be coated with insulation material 220, 220a, 220b (not shown), 220c (not shown) and 220d, which has second electrical conductivity. It is shown that the electroacupuncture needle 200 is coated with the one layer of insulation material 220 and includes two boundary lines having discontinuous conductivity. It is also shown that the electroacupuncture needle 200a, 200b, 200c and 200d is coated with the insulation material 220a, 220b (not shown), 220c (not shown) and 220d in the form of multi-layers in a variety of shapes and has more than two boundary lines having discontinuous electrical conductivity. Here, the boundary line may be horizontal or diagonal.

FIG. 3 shows that electroacupuncture needle 300a and 300b is implemented by plating in accordance with the embodiment of the present invention. In FIG. 3, the electroacupuncture needle 300a may be formed with a material having the first electrical conductivity. The electroacupuncture needle 300a may be plated with a metallic material having the second electrical conductivity different from the first electrical conductivity. For example, it is shown that the body of the electroacupuncture needle 300a is plated with platinum 310a, gold 320a and silver 330a one by one. However, this is only an example. It is possible that one metallic material having the second electrical conductivity is plated on the electroacupuncture needle 300a or another kind of a metallic material is plated on the electroacupuncture needle 300a. Also, a larger number of the metallic materials may be plated. As a result, at least two boundary lines having discontinuous electrical conductivity may be formed in the body of the electroacupuncture needle 300a.

In FIG. 3, the electroacupuncture needle 300b is formed with a material having the first electrical conductivity, and metal particles having the second electrical conductivity different from the first electrical conductivity are attached to the body of the electroacupuncture needle 300b. That is, a metallic material having the second electrical conductivity is not uniformly plated on the body of the electroacupuncture needle and the metal particles are individually attached to the electroacupuncture needle 300b. Such a method forms the unevenness on the surface of the body rather than the boundary line having discontinuous electrical conductivity in the body.

FIG. 4 shows that an electroacupuncture needle 400 is implemented by changing a constituent metallic material in accordance with the embodiment of the present invention. That is, the body of the electroacupuncture needle 400 may be made of metallic material layers which have mutually different electrical conductivities. For instance, the body of the electroacupuncture needle 400 may be made of a platinum layer 410, a gold layer 420 and a silver layer 430. In FIG. 4, it is possible to form the boundary line having discontinuous electrical conductivity by changing the material layer itself constituting the body of the electroacupuncture needle 400 instead of coating metallic materials having mutually different electrical conductivities on the surface of the electroacupuncture needle. While FIG. 4 shows that three metallic material layers are included in the body, this is just an example. It is also possible to form the electroacupuncture needle 400 having two or more boundary lines by use of the material constituting the electroacupuncture needle 400 and one or more material having mutually different electrical conductivities. Additionally, metallic materials other than platinum, gold and silver can be used.

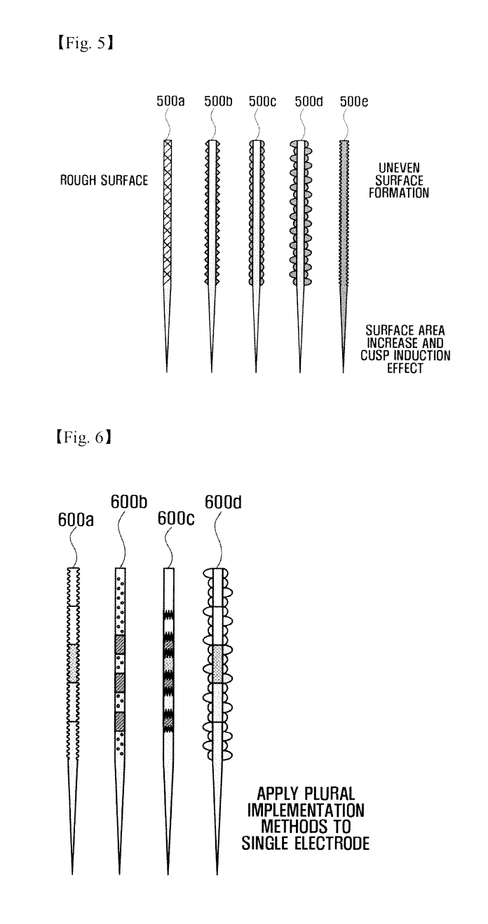

FIG. 5 shows an example in which unevenness functioning as a cusp is formed on the surfaces of the body of electroacupuncture needle 500a, 500b, 500c, 500d and 500e. In FIG. 5, the surface of the electroacupuncture needle 500a is textured. The unevenness can be formed by roughing the surface of the body of the electroacupuncture needle. Also, the unevenness is formed on the surface of the electroacupuncture needle 500b, 500c, 500d and 500e. The unevenness can be formed by making the circumference of the body uneven or by making the surface of the body uneven such that the center of the circumference of the body is not consistent over the length of the entire body. In addition, unevenness functioning as a cusp on which electric charges are concentrated on the surface of the body of the electroacupuncture needle can be implemented in various ways.

FIG. 6 shows that electroacupuncture needle 600a, 600b, 600c and 600d is implemented by complexly using the methods of FIGS. 2 to 5 in accordance with the embodiment of the present invention. That is, in FIG. 6, the electroacupuncture needle 600a is implemented by combining the implementation method of electroacupuncture needle 500 in FIG. 5 and the implementation methods of electroacupuncture needles 300a and 400 in FIG. 3 or 4. In FIG. 6, the electroacupuncture needle 600b is implemented by combining the implementation method of electroacupuncture needle 200a in FIG. 2 and the implementation method of electroacupuncture needle 300b in FIG. 3. In FIG. 6, the electroacupuncture needle 600c is implemented by combining the implementation method of electroacupuncture needle 200d in FIG. 2 and the implementation method of electroacupuncture needle 300a or 400 in FIG. 3 or 4. In FIG. 6, the electroacupuncture needle 600d is implemented by combining the implementation method of electroacupuncture needle 500d in FIG. 5 and the implementation method of electroacupuncture needle 300a or 400 in FIG. 3 or 4. Besides the embodiments shown in FIG. 6, the boundary line having discontinuous electrical conductivity and/or the unevenness can be formed on the body of the electroacupuncture needle by complexly using other methods.

An electroacupuncture platform according to the embodiment of the present invention includes a current supply (not shown) supplying current for electrical stimulation and includes a plurality of electroacupuncture needles electrically connected to one end of the current supply. At least one of the plurality of the electroacupuncture needles may be the electroacupuncture needle according to the embodiment of the present invention. As such, the electroacupuncture platform uses the electroacupuncture needle according to the embodiment of the present invention, so that it is possible to control or induce a current path between the electroacupuncture needles inside the skin of the subject.

Here, the current supply is able to supply electrical stimulation current to the electroacupuncture needle. The electrical stimulation current may have a pulse type. The waveform and intensity of the pulse may be optionally controlled according to the embodiment of the present invention. The electroacupuncture platform according to the embodiment of the present invention is connected to the other end of the current supply and may further include a ground electrode contacting with the skin of the subject. The following embodiments show that an electroacupuncture needle is used as the ground electrode. Also, the electroacupuncture platform according to the embodiment of the present invention may further include a power supply (not shown) supplying energy required for the electrical stimulation.

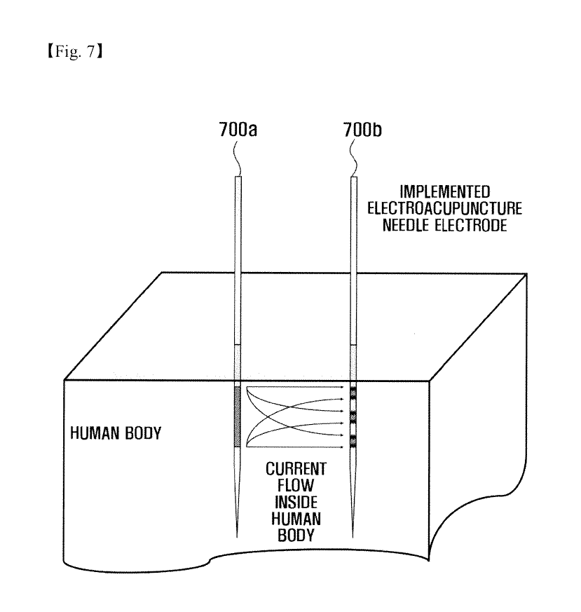

FIG. 7 shows a current path induction effect of an electroacupuncture platform using two electroacupuncture needles 700a and 700b according to the embodiment of the present invention. Each of the two electroacupuncture needles 700a and 700b includes the boundary line having discontinuous electrical conductivity. Here, unlike a case where a general electroacupuncture needle is used, it can be seen that current flow is concentrated on the boundary lines of the electroacupuncture needles 700a and 700b, which have discontinuous electrical conductivity.

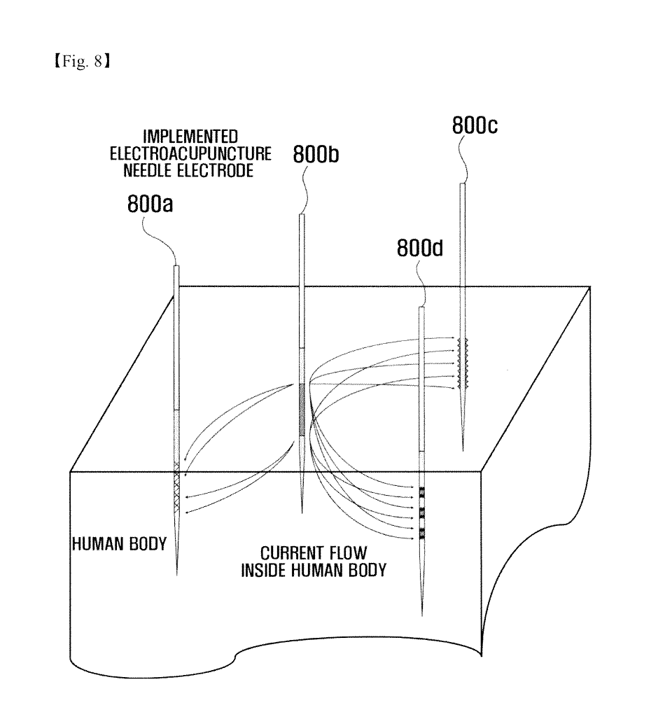

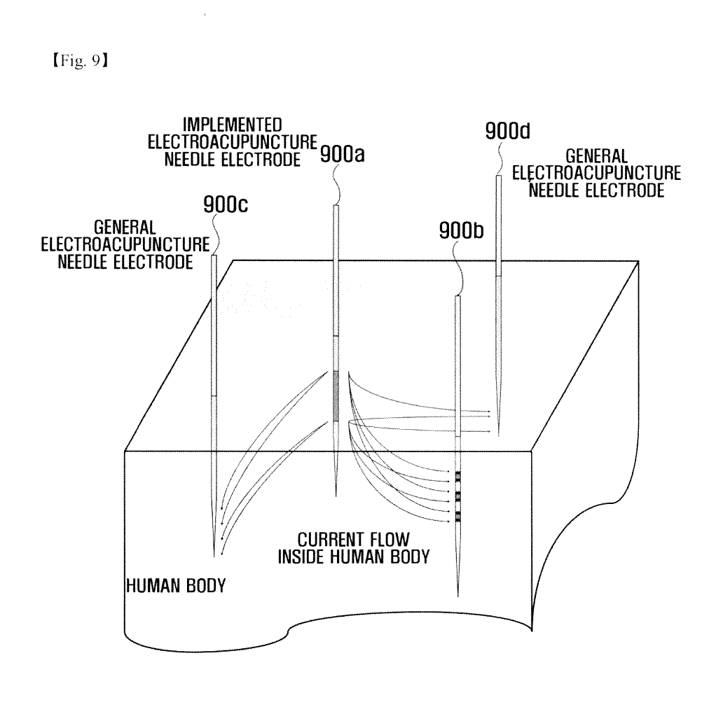

FIG. 8 shows a current path induction effect of the electroacupuncture platform using four electroacupuncture needles 800a, 800b, 800c and 800d according to the embodiment of the present invention. It is shown that each of the electroacupuncture needles 800b and 800d includes the boundary line having discontinuous electrical conductivity, and each of the surfaces of the electroacupuncture needles 800a and 800c has unevenness formed thereon. Here, unlike a case where a general electroacupuncture needle is used, it can be seen that current flow is concentrated on the boundary lines of the electroacupuncture needles 800b and 800d and on the unevenness functioning as a cusp of the electroacupuncture needles 800a and 800c.

FIG. 9 shows a current path induction effect of the electroacupuncture platform including two general electroacupuncture needles 900c and 900d and two electroacupuncture needles 900a and 900b according to the embodiment of the present invention. It is shown that each of the electroacupuncture needles 900a and 900b includes the boundary line having discontinuous electrical conductivity. Each of the electroacupuncture needles 900c and 900d is a general electroacupuncture needle which has neither discontinuous electrical conductivity nor unevenness formed thereon. It can be seen that while the fact that current flow is concentrated on the pointed ends of the general electroacupuncture needles 900c and 900d, the current flow is concentrated on the boundary lines of the electroacupuncture needles 900a and 900b, which have discontinuous electrical conductivity.

As such, as the electroacupuncture needle according to the embodiment of the present invention is used in the electroacupuncture platform, it is possible to expect and/or induce a current flow path between the electroacupuncture needles. In other words, since it is possible to recognize where the current flow is concentrated on in the electroacupuncture needle according to the embodiment of the present invention, the current can be induced to a required tissue portion of the subject. It is possible to variously control the current flow path between the electroacupuncture needles through an appropriate combination of a general electroacupuncture needle and the electroacupuncture needle according to the embodiment of the present invention if necessary.

Further, in the electroacupuncture needle according to the embodiment of the present invention, electric charges are concentrated on the cusp of the unevenness and the boundary line having discontinuous electrical conductivity. Therefore, an electrical resistance to the current flow between the electroacupuncture needles may be reduced in the use of the electroacupuncture needle according to the embodiment of the present invention. As a result, a better therapeutic effect caused by a small amount of energy can be obtained by using the electroacupuncture needle according to the embodiment of the present invention.

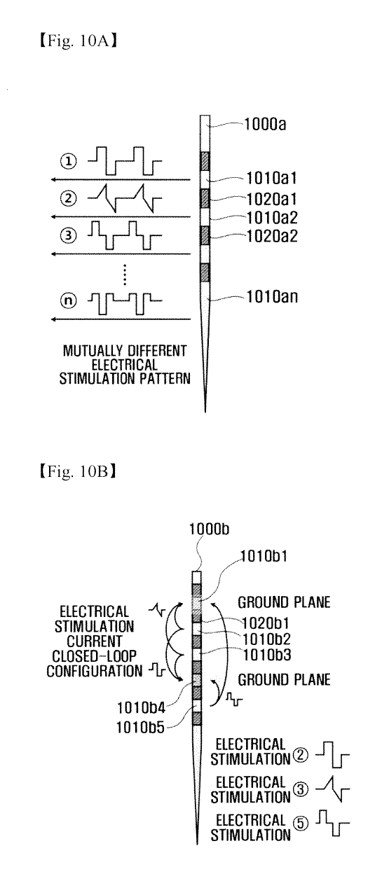

Additionally, mutually different electrical stimulation patterns can be applied to the electroacupuncture needle according to the embodiment of the present invention. FIG. 10A shows how to apply mutually different electrical stimulation patterns to a plurality of electrical conductive surfaces of one electroacupuncture needle in accordance with the embodiment of the present invention. As shown in FIG. 10A, the body of an electroacupuncture needle 1000a may include a plurality of electrical conductive surfaces 1010a1, 1010a2, . . . , 1010an which are electrically insulated from each other by insulation materials 1020a1, 1020a2, . . . , 1020an.

The electroacupuncture needle 1000a may be, as shown in FIG. 2, formed by coating the insulation material on an electrical conducting material. Besides, through a combination of the insulation material coating method of FIG. 2, the plating method of FIG. 3 and/or the constituent metallic material alternation method of FIG. 4, it is possible to implement the electroacupuncture needle including a plurality of electrical conductive surfaces which are electrically insulated from each other by the insulation materials.

FIG. 10A shows that mutually different electrical stimulation patterns are respectively applied to the plurality of the electrical conductive surfaces 1010a1, . . . , 1010a2, . . . , 1010an which are electrically insulated from each other by the insulation materials 1020a1, 1020a2, . . . , 1020an. However, this is only an example and means that the electrical stimulation pattern may be individually (or independently) applied to each of the electrical conductive surfaces 1010a1, 1010a2, . . . 1010an. That is, it is possible that the same electrical stimulation pattern may be applied to all of the plurality of the electrical conductive surfaces 1010a1, 1010a2, . . . , 1010an or it is also possible that mutually different electrical stimulation patterns may be applied to two or more electrical conductive surfaces. Here, an individual wire may be connected to the plurality of the electrical conductive surfaces 1010a1, 1010a2, . . . , 1010an respectively, and then the electrical stimulation pattern may be independently applied to each of the electrical conductive surfaces 1010a1, 1010a2, . . . , 1010an.

Regarding an electroacupuncture needle 1000b according to the embodiment of the present invention, while the electrical stimulation pattern is applied to some of the electrical conductive surfaces, at least one electrical conductive surface may be used as a ground plane. FIG. 10B shows how at least one electrical conductive surface of one electroacupuncture needle is used as ground planes 1010b1 and 1010b4 in accordance with the embodiment of the present invention.

As shown in FIG. 10B, the electrical stimulation pattern may be individually applied to three 1010b2, 1010b3 and 1010b5 of a plurality of electrical conductive surfaces 1010b1, 1010b2, 1010b3, 1010b4 and 1010b5 which are electrically insulated from each other by insulation materials 1020b1, 1020b2, . . . , 1020bn, and the other two electrical conductive surfaces 1010b1 and 1010b4 may be used as a ground. In this manner, at least one of a plurality of the electrical conductive surfaces 1010b1, 1010b2, 1010b3, 1010b4 and 1010b5 of the one electroacupuncture needle 1000b is used as a ground plane, and the electrical stimulation pattern is individually applied to the other electrical conductive surfaces, so that a closed loop of stimulation current required for the electrical stimulation can be configured in the one electroacupuncture needle 1000b.

As shown in FIGS. 10A and 10B, the electrical stimulation pattern is individually applied on each of the plurality of the electrical conductive surfaces of one electroacupuncture needle or at least one of the plurality of the electrical conductive surfaces is used as a ground plane. As a result, a random electrical stimulation effect can be obtained if necessary.

The electroacupuncture needle shown in FIGS. 10A and 10B may be individually used in the electroacupuncture platform or may be used together with the plural electroacupuncture needles in the electroacupuncture platform shown in FIGS. 7 to 9.

While the embodiment of the present invention has been described with reference to the accompanying drawings, it can be understood by those skilled in the art that the present invention can be embodied in other specific forms without departing from s spirit or essential characteristics. Therefore, the foregoing embodiments and advantages are merely exemplary and are not to be construed as limiting the present invention. The present teaching can be readily applied to other types of apparatuses. The description of the foregoing embodiments is intended to he illustrative, and not to limit the scope of the claims. Many alternatives, modifications, and variations will be apparent to those skilled in the art. In the claims, means-plus-function clauses are intended to cover the structures described herein as performing the recited function and not only structural equivalents but also equivalent structures.

1. An electroacupuncture needle comprising:
a body which includes at least one boundary line having discontinuous electrical conductivity; and
an end which is formed to have a pointed shape allowing the electroacupuncture needle to be easily inserted into the skin of a subject.

2. The electroacupuncture needle of claim 1, wherein the boundary line is formed by coating an insulation material on a portion of the body.

3. The electroacupuncture needle of claim 1, wherein the boundary line is formed by plating a metallic material having electrical conductivity different from the electrical conductivity of the body.

4. The electroacupuncture needle of claim 1, wherein the body is configured with metallic material layers which have mutually different electrical conductivities, so that the boundary line is formed.

5. The electroacupuncture needle of claim 1, wherein unevenness is formed on the surface of the body.

6. The electroacupuncture needle of claim 1, wherein the body comprises a plurality of electrical conductive surfaces which are electrically insulated from each other by an insulation material, and wherein mutually different electrical stimulation patterns are applied to at least two of the plurality of the electrical conductive surfaces.

7. The electroacupuncture needle of claim 1, wherein the body comprises a plurality of electrical conductive surfaces which are electrically insulated from each other by an insulation material, and wherein at least one of the plurality of the electrical conductive surfaces is used as a ground plane.

8. An electroacupuncture needle comprising:
a body which has unevenness fowled on the surface thereof; and
an end which is formed to have a pointed shape allowing the electroacupuncture to be easily inserted into the skin of a subject.

9. The electroacupuncture needle of claim 8, wherein the unevenness is formed by attaching metal particles having electrical conductivity different from the electrical conductivity of the body to the body.

10. The electroacupuncture needle of claim 8, wherein the unevenness is formed by making the circumference of the body uneven or by making the surface of the body uneven such that the center of the circumference of the body is not consistent.

11. An electroacupuncture platform in comprising:
a current supply supplying current for electrical stimulation; and
a plurality of electroacupuncture needles electrically connected to one end of the current supply,
wherein at least one of the plurality of the electroacupuncture needles includes:
a body which includes at least one boundary line having discontinuous electrical conductivity; and
an end which is formed to have a pointed shape allowing the electroacupuncture needle to be easily inserted into the skin of a subject.

12. The electroacupuncture platform of claim 11 comprising:
a electroacupuncture needle wherein the boundary line is formed by coating an insulation material on a portion of the body.

13. The electroacupuncture platform of claim 11 comprising:
a electroacupuncture needle wherein the boundary line is formed by plating a metallic material having electrical conductivity different from the electrical conductivity of the body.

14. The electroacupuncture platform of claim 11 comprising:
a electroacupuncture needle wherein the body is configured with metallic material layers which have mutually different electrical conductivities, so that the boundary line is formed.

15. The electroacupuncture platform of claim 11 comprising:
a electroacupuncture needle wherein unevenness is formed on the surface of the body.

16. The electroacupuncture platform of claim 11 comprising:
a electroacupuncture needle wherein the body comprises a plurality of electrical conductive surfaces which are electrically insulated from each other by an insulation material, and wherein mutually different electrical stimulation patterns are applied to at least two of the plurality of the electrical conductive surfaces.

17. The electroacupuncture platform of claim 11 comprising:
a electroacupuncture needle wherein the body comprises a plurality of electrical conductive surfaces which are electrically insulated from each other by an insulation material, and wherein at least one of the plurality of the electrical conductive surfaces is used as a ground plane.

18. The electroacupuncture platform of claim 11 comprising:
a body which has unevenness formed on the surface thereof; and
an end which is formed to have a pointed shape allowing the electroacupuncture to be easily inserted into the skin of a subject.

19. The electroacupuncture platform of claim 11 comprising:
a electroacupuncture needle wherein the unevenness is formed by attaching metal particles having electrical conductivity different from the electrical conductivity of the body to the body.

20. The electroacupuncture platform of claim 11 comprising:
a electroacupuncture needle wherein the unevenness is formed by making the circumference of the body uneven or by making the surface of the body uneven such that the center of the circumference of the body is not consistent.
